(19) United States
(12) Patent Application Publication
(10) Pub. No.: US 2020/0024352 A1
(43) Pub. Date: Jan. 23, 2020

(54) ANTI-TIM-3 ANTIBODIES FOR COMBINATION WITH ANTI-PD-L1 ANTIBODIES

(71) Applicant: Eli Lilly and Company, Indianapolis, IN (US)

(72) Inventors: Yiwen LI, Woodcliff, NJ (US); Yi ZHANG, Edison, NJ (US)

(21) Appl. No.: 16/346,769

(22) PCT Filed: Dec. 1, 2017
(86) PCT No.: PCT/US17/64207

§ 371 Date: May 1, 2019

Related U.S. Application Data
(60) Provisional application No. 62/431,485, filed on Dec. 8, 2016.

(51) Int. Cl. C07K 16/28; A61P 35/00

(52) U.S. Cl. CPC C07K 16/2827; C07K 2317/92; C07K 2317/565; A61P 35/00

(57) ABSTRACT

The present invention relates to antibodies that bind human T-cell immunoglobulin- and mucin-domain-containing protein-3 (Tim-3), and may be useful for treating solid and hematological tumors in combination with anti-human PD-L1 antibodies, chemotherapy, and ionizing radiation.

The present invention is in the field of medicine. Particularly, the present invention relates to antibodies directed to human T-cell immunoglobulin- and mucin-domain-containing protein-3 (Tim-3) that can be combined with antibodies directed to human PD-L1, compositions comprising such anti-human Tim-3 antibodies or anti-human PD-L1 antibodies, and methods of using such anti-human Tim-3 antibodies in combination with anti-human PD-L1 antibodies for the treatment of solid and hematological tumors alone or in further combination with chemotherapy, ionizing radiation, and other cancer therapeutics.

Tumor cells escape detection and elimination by the immune system through multiple mechanisms some of which include the manipulation of immune checkpoint pathways. Immune checkpoint pathways are used in self-tolerance maintenance and in the regulation of T cell activation, but cancer cells can manipulate these pathways to prolong tumor survival. The PD-1/human programmed cell death 1 ligand 1 (PD-L1) pathway is one such immune checkpoint. Human PD-1 is expressed on T cells, and the binding of PD-L1 or PD-L2 to PD-1 has been shown to inhibit T cell proliferation and cytokine production. Moreover, some tumors are known to express PD-L1 and PD-L2 and such expression can contribute to the inhibition of the intratumoral immune response.

In addition to the PD-1/PD-L1 pathway, T cells recognizing tumor antigens can also express other checkpoint receptors, such as Tim-3. In particular, T cells expressing Tim-3 can exhibit an exhausted phenotype characterized by an impairment in cytotoxic functions, effector cytokine production, and proliferation. In this regard, it has been shown that anti-Tim-3 antibodies can restore anti-tumor immunity in some murine cancer models. Moreover, it has also been shown that some patients who develop adaptive resistance to anti-PD-1 treatment display an upregulation of Tim-3 on their T cells.

Antibodies directed to human Tim-3 are known. Humanized antibodies against human Tim-3 are described in WO15117002. MBG453, an anti-human Tim-3 antibody, is currently being tested in human clinical trials as a single agent and in combination with an anti-human PD-1 antibody. However, no antibody targeting Tim-3 has been approved for therapeutic use in humans nor has any anti-human Tim-3 antibody been shown to display enhanced efficacy when combined with an anti-human PD-L1 antibody. Thus, there remains a need for anti-human Tim-3 antibodies that can be combined with anti-human PD-L1 antibodies as well as other therapies for treating human cancers.

Tim-3 (SEQ ID NO:1) has been shown to interact with galectin-9 (SEQ ID NO:15), phosphaditylserine (C₁₃H₂₄NO₁₀P), high-mobility group Box 1 (HMGB1), and carcinoembryonic antigen cell adhesion molecule 1(CEACAM1) (SEQ ID NO:14). Because all of the aforementioned Tim-3 ligands are not exclusive ligands of Tim-3, it is desirable to provide therapeutic anti-Tim-3 antibodies that differentially block the activity of said ligands as these ligands can regulate the immune system independently of Tim-3. Such a strategy can provide alternative ways to more specifically modulate Tim-3 activity, allowing for tailored immuno-oncology based therapies for patients. Furthermore, such anti-Tim-3 antibodies can provide options for combinatorial therapies with anti-human PD-L1 antibodies. Thus, there also remains a need to provide antibodies that bind human Tim-3 and inhibit Tim-3's interactions with some of Tim-3's ligands, but not others, and that can be combined with anti-human PD-L1 antibodies.

The anti-human Tim-3 antibodies described herein can block human Tim-3 (SEQ ID NO: 1) from binding to human galectin-9 (SEQ ID NO:15) and phosphatidylserine while simultaneously not blocking the binding of human Tim-3 and human CEACAM1 (SEQ ID NO:14) and may be combined with anti-human PD-L1 antibodies for the treatment of cancer.

While antibodies targeting PD-L1 (SEQ ID NO:16) for cancer immunotherapy have proven effective for some cancers, some cancers become less sensitive to PD-L1 therapy over time or do not respond at all. In some embodiments, the present invention provides an anti-human Tim-3 antibody that can be administered to patients who have progressed or are progressing under anti-human PD-L1 antibody therapy. In some embodiments, the present invention provides an anti-human Tim-3 antibody that can be administered in combination with an anti-human PD-L1 antibody to patients who have not previously received anti-human PD-L1 antibody therapy.

The present invention includes anti-human Tim-3 (SEQ ID NO: 1) antibodies comprising HCDR1, HCDR2, HCDR3, LCDR1, LCDR2, and LCDR3 consisting of the amino acid sequences of SEQ ID NOs: 2, 3, 4, 5, 6, and 7, respectively; a heavy chain variable region having the amino acid sequence of SEQ ID NO: 8 and a light chain variable region having the amino acid sequence of SEQ ID NO: 9; and/or a heavy chain having the amino acid sequence of SEQ ID NO: 10 and a light chain having the amino acid sequence of SEQ ID NO: 11 for simultaneous, separate, or sequential combination with an anti-human PD-L1 antibody.

Non-limiting examples of known anti-human PD-L1 antibodies include atezolizumab, durvalumab, avelumab, and BMS-936559. It is to be recognized that atezolizumab, durvalumab, avelumab, and BMS-936559, as used herein, can be made using a variety of cell lines and using various manufacturing processes and may exhibit some differences as a result. Atezolizumab is an antibody comprising the light chain having the amino acid sequence of SEQ ID NO: 29 and heavy chain having the amino acid sequence of SEQ ID NO: 30. Durvalumab is an antibody comprising the light chain having the amino acid sequence of SEQ ID NO: 31 and heavy chain having the amino acid sequence of SEQ ID NO: 32. Avelumab is an antibody comprising the light chain having the amino acid sequence of SEQ ID NO: 33 and heavy chain having the amino acid sequence of SEQ ID NO: 34. BMS-936559 is an antibody, preferably a fully human IgG4 antibody, comprising the light chain variable region (LCVR) having the amino acid sequence of SEQ ID NO: 35 and heavy variable region (HCVR) having the amino acid sequence of SEQ ID NO: 36.

Non-limiting examples of other anti-human PD-L1 (SEQ ID NO:16) antibodies include anti-human PD-L1 antibodies comprising one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises comprises a heavy chain having the amino acid sequence of SEQ ID NO: 10 and a light chain having the amino acid sequence of SEQ ID NO: 11.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody is BMS-936559, atezolizumab, durvalumab, or avelumab.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody is BMS-936559, atezolizumab, durvalumab, or avelumab.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises comprises a heavy chain having the amino acid sequence of SEQ ID NO: 10 and a light chain having the amino acid sequence of SEQ ID NO: 11; wherein the anti-human PD-L1 antibody is BMS-936559, atezolizumab, durvalumab, or avelumab.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; (c) a heavy chain having the amino acid sequence of SEQ ID NO: 25 and a light chain having the amino acid sequence of SEQ ID NO: 26.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises: a heavy chain variable region having the amino acid sequence of SEQ ID NO: 23 and a light chain having the amino acid sequence of SEQ ID NO: 24.

A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain having the amino acid sequence of SEQ ID NO: 25 and a light chain having the amino acid sequence of SEQ ID NO: 26.

A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is lung cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the lung cancer is non-small cell lung cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is head and neck cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is colorectal cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is pancreatic cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is gastric cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is kidney cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is bladder cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is prostate cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is breast cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is ovarian cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is esophageal cancer. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is soft tissue sarcoma. A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is liver cancer.

A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; wherein at least one of the anti-human Tim-3 antibody and anti-human PD-L1 antibody is administered in simultaneous, separate, or sequential combination with ionizing radiation and/or one or more chemotherapeutic agents.

A method of treating cancer comprising administering to a patient in need, thereof an effective amount of an anti-human Tim-3 (SEQ ID NO: 1) antibody of the present invention in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26; wherein at least one of the anti-human Tim-3 antibody and anti-human PD-L1 antibody is administered in simultaneous, separate, or sequential combination with ionizing radiation and/or one or more chemotherapeutic agents.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises comprises a heavy chain having the amino acid sequence of SEQ ID NO: 10 and a light chain having the amino acid sequence of SEQ ID NO: 11.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody is BMS-936559, atezolizumab, durvalumab, or avelumab.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody is BMS-936559, atezolizumab, durvalumab, or avelumab.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises comprises a heavy chain having the amino acid sequence of SEQ ID NO: 10 and a light chain having the amino acid sequence of SEQ ID NO: 11; wherein the anti-human PD-L1 antibody is BMS-936559, atezolizumab, durvalumab, or avelumab.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody comprises at least one of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody comprises a heavy chain having the amino acid sequence of SEQ ID NO: 25 and a light chain having the amino acid sequence of SEQ ID NO: 26.

An anti-human Tim-3 (SEQ ID NO: 1) antibody of the present invention for use in simultaneous, separate, or sequential combination with an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain having the amino acid sequence of SEQ ID NO: 25 and a light chain having the amino acid sequence of SEQ ID NO: 26.

An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is lung cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the lung cancer is non-small cell lung cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is head and neck cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is colorectal cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is pancreatic cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is gastric cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is kidney cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is bladder cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is prostate cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is breast cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is ovarian cancer. An effective amount of an anti-human Tim-3 (SEQ ID NO: 1) antibody of the present invention for use in simultaneous, separate, or sequential combination with an effective amount of an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is esophageal cancer. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is soft tissue sarcoma. An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; and wherein the cancer is liver cancer.

An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; wherein at least one of the anti-human Tim-3 antibody and anti-human PD-L1 antibody is administered in simultaneous, separate, or sequential combination with ionizing radiation and/or one or more chemotherapeutic agents.

An anti-human Tim-3 (SEQ ID NO: 1) antibody of the present invention for use in simultaneous, separate, or sequential combination with an anti-human PD-L1 (SEQ ID NO:16) antibody; optionally, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26; wherein at least one of the anti-human Tim-3 antibody and anti-human PD-L1 antibody is administered in simultaneous, separate, or sequential combination with ionizing radiation and/or one or more chemotherapeutic agents.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain variable region having the amino acid sequence of SEQ ID NO: 8 and a light chain variable region having the amino acid sequence of SEQ ID NO: 9.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain having the amino acid sequence of SEQ ID NO: 10 and a light chain having the amino acid sequence of SEQ ID NO: 11.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody is BMS-936559, atezolizumab, durvalumab, or avelumab.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody is BMS-936559, atezolizumab, durvalumab, or avelumab.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises comprises a heavy chain having the amino acid sequence of SEQ ID NO: 10 and a light chain having the amino acid sequence of SEQ ID NO: 11; wherein the anti-human PD-L1 antibody is BMS-936559, atezolizumab, durvalumab, or avelumab.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain variable region having the amino acid sequence of SEQ ID NO: 8 and a light chain variable region having the amino acid sequence of SEQ ID NO: 9; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain variable region having the amino acid sequence of SEQ ID NO: 23 and a light chain variable region having the amino acid sequence of SEQ ID NO: 24.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain having the amino acid sequence of SEQ ID NO: 25 and a light chain having the amino acid sequence of SEQ ID NO: 26.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is lung cancer. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is melanoma. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the lung cancer is non-small cell lung cancer. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is head and neck cancer. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody, optionally, wherein the anti-human Tim-3 antibody comprises a heavy chain having the amino acid sequence of SEQ ID NO: 10 and a light chain having the amino acid sequence of SEQ ID NO: 11; and wherein the cancer is colorectal cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is pancreatic cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is gastric cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is kidney cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is bladder cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is prostate cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is breast cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is ovarian cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is esophageal cancer. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is soft tissue sarcoma. An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; and wherein the cancer is liver cancer.

An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; wherein at least one of the anti-human Tim-3 antibody and anti-human PD-L1 antibody is administered in simultaneous, separate, or sequential combination with ionizing radiation and/or one or more chemotherapeutic agents.

An anti-human Tim-3 (SEQ ID NO:1) antibody and an anti-human PD-L1 (SEQ ID NO:16) antibody for the manufacture of a medicament for the treatment of cancer, optionally, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26; wherein at least one of the anti-human Tim-3 antibody and anti-human PD-L1 antibody is administered in simultaneous, separate, or sequential combination with ionizing radiation and/or one or more chemotherapeutic agents.

A kit for the treatment of cancer, the kit comprising a first pharmaceutical composition comprising an anti-human Tim-3 (SEQ ID NO:1) antibody of the present invention and a second pharmaceutical composition comprising an anti-human PD-L1 (SEQ ID NO:16) antibody. A kit for the treatment of cancer, the kit comprising a first pharmaceutical composition comprising an anti-human Tim-3 (SEQ ID NO:1) antibody of the present invention and a second pharmaceutical composition comprising an anti-human PD-L1 (SEQ ID NO:16) antibody; wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

A kit for the treatment of cancer, the kit comprising a first pharmaceutical composition comprising an anti-human Tim-3 (SEQ ID NO:1) antibody and a second pharmaceutical composition comprising an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11.

A kit for the treatment of cancer, the kit comprising a first pharmaceutical composition comprising an anti-human Tim-3 (SEQ ID NO:1) antibody and a second pharmaceutical composition comprising an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody is BMS-936559, atezolizumab, durvalumab, or avelumab.

A kit for the treatment of cancer, the kit comprising a first pharmaceutical composition comprising an anti-human Tim-3 (SEQ ID NO:1) antibody and a second pharmaceutical composition comprising an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

A kit for the treatment of cancer, the kit comprising a first pharmaceutical composition comprising an anti-human Tim-3 (SEQ ID NO:1) antibody and a second pharmaceutical composition comprising an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain variable region having the amino acid sequence of SEQ ID NO: 23 and a light chain variable region having the amino acid sequence of SEQ ID NO: 24.

A kit for the treatment of cancer, the kit comprising a first pharmaceutical composition comprising an anti-human Tim-3 (SEQ ID NO:1) antibody and a second pharmaceutical composition comprising an anti-human PD-L1 (SEQ ID NO:16) antibody; wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain having the amino acid sequence of SEQ ID NO: 25 and a light chain having the amino acid sequence of SEQ ID NO: 26.

An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14). An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human Tim-3 antibody also blocks binding of human Tim-3 to human galectin-9 (SEQ ID: 15).

An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer. An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human Tim-3 antibody also blocks binding of human Tim-3 to human galectin-9 (SEQ ID: 15); wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human PD-L1 antibody is BMS-936559, atezolizumab, durvalumab, or avelumab. An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human Tim-3 antibody also blocks binding of human Tim-3 to human galectin-9 (SEQ ID: 15); wherein the anti-human PD-L1 antibody is BMS-936559, atezolizumab, durvalumab, or avelumab.

An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26. An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody blocks binding of human Tim-3 to human phosphatidylserine, but does not block binding of human Tim-3 to human CEACAM1 (SEQ ID: 14); wherein the anti-human Tim-3 antibody also blocks binding of human Tim-3 to human galectin-9 (SEQ ID: 15); wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody contacts at least one amino acid residue of the following on human Tim-3 (SEQ ID NO:1): 50, 55, 62-65 (inclusive), 72, 111, and 113-118 (inclusive). An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody contacts at least one amino acid residue of the following on human Tim-3 (SEQ ID NO:1): 50, 55, 62-65 (inclusive), 72, 111, and 113-118 (inclusive); wherein the anti-human Tim-3 antibody contacts: at least two of the residues; preferably at least three of the residues; more preferably at least four of the residues; more preferably at least five of the residues; more preferably at least six of the residues; more preferably at least seven of the residues; more preferably at least eight of the residues; more preferably at least nine of the residues; more preferably at least ten of the residues; more preferably at least eleven of the residues; more preferably at least twelve of the residues; more preferably at least thirteen of the residues; or more preferably all of the residues. An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody contacts at least one amino acid residue of the following on human Tim-3 (SEQ ID NO:1): 50, 55, 62-65 (inclusive), 72, 111, and 113-118 (inclusive); wherein the anti-human Tim-3 antibody contacts: at least two of the residues; preferably at least three of the residues; more preferably at least four of the residues; more preferably at least five of the residues; more preferably at least six of the residues; more preferably at least seven of the residues; more preferably at least eight of the residues; more preferably at least nine of the residues; more preferably at least ten of the residues; more preferably at least eleven of the residues; more preferably at least twelve of the residues; more preferably at least thirteen of the residues; or more preferably all of the residues; wherein the anti-human Tim-3 antibody further contacts at least one residue of the following: 56-61 (inclusive), 107, 119-120 (inclusive), and 122. An anti-human Tim-3 (SEQ ID NO:1) antibody for use in simultaneous, separate, or sequential combination with an anti-human PD-L1 (SEQ ID NO:16) antibody, in the treatment of cancer, wherein the anti-human Tim-3 antibody contacts at least one amino acid residue of the following on human Tim-3 (SEQ ID NO:1): 50, 55, 62-65 (inclusive), 72, 111, and 113-118 (inclusive); wherein the anti-human Tim-3 antibody contacts: at least two of the residues; preferably at least three of the residues; more preferably at least four of the residues; more preferably at least five of the residues; more preferably at least six of the residues; more preferably at least seven of the residues; more preferably at least eight of the residues; more preferably at least nine of the residues; more preferably at least ten of the residues; more preferably at least eleven of the residues; more preferably at least twelve of the residues; more preferably at least thirteen of the residues; or more preferably all of the residues; wherein the anti-human Tim-3 antibody further contacts at least one residue of the following: 56-61 (inclusive), 107, 119-120 (inclusive), and 122; wherein the residues in contact are within six (6) angstroms or less of the anti-human Tim-3 antibody, as determined by X-ray crystallography.

A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11. A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody is atezolizumab, durvalumab, avelumab, or BMS-936559. A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain variable region having the amino acid sequence of SEQ ID NO: 8 and a light chain variable region having the amino acid sequence of SEQ ID NO: 9. A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises one or more of the following: (a) HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22; (b) a heavy chain variable region having the amino acid sequence of SEQ ID NO: 23 and a light chain variable region having the amino acid sequence of SEQ ID NO: 24; and (c) a heavy chain having the amino acid sequence of SEQ ID NO: 25 and a light chain having the amino acid sequence of SEQ ID NO: 26.

A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain variable region having the amino acid sequence of SEQ ID NO: 23 and a light chain variable region having the amino acid sequence of SEQ ID NO: 24.

A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11; wherein the anti-human PD-L1 antibody comprises a heavy chain having the amino acid sequence of SEQ ID NO: 25 and a light chain having the amino acid sequence of SEQ ID NO: 26.

A first pharmaceutical composition comprising an anti-human Tim-3 (SEQ ID NO:1) antibody for use in simultaneous, separate, or sequential combination with a second pharmaceutical composition comprising an anti-human PD-L1 (SEQ ID NO:16) antibody, in the treatment of cancer, wherein the anti-human Tim-3 antibody comprises a heavy chain having the amino acid sequence of SEQ ID NO: 10 and a light chain having the amino acid sequence of SEQ ID NO: 11; wherein either the anti-human PD-L1 antibody comprises a heavy chain having the amino acid sequence of SEQ ID NO: 25 and a light chain having the amino acid sequence of SEQ ID NO: 26 or is atezolizumab, durvalumab, avelumab, or BMS-936559.

The antibodies of the present invention are engineered, non-naturally occurring polypeptide complexes. A DNA molecule of the present invention is a non-naturally occurring DNA molecule that comprises a polynucleotide sequence encoding a polypeptide having the amino acid sequence of one of the polypeptides in an antibody of the present invention.

The antibodies of the present invention are an IgG type antibody and have two “heavy” chains and two “light” chains that are cross-linked via intra- and inter-chain disulfide bonds. Each heavy chain is comprised of an N-terminal HCVR and a heavy chain constant region (“HCCR”) and has the same amino acid sequence. Each light chain is comprised of a LCVR and a light chain constant region (“LCCR”) and has the same amino acid sequence. When expressed in certain biological systems, antibodies having native human Fc sequences are glycosylated in the Fc region. Typically, glycosylation occurs in the Fc region of the antibody at a highly conserved N-glycosylation site. N-glycans typically attach to asparagine. Antibodies may be glycosylated at other positions as well.

Optionally, certain anti-Tim-3 antibodies described herein contain an Fc portion that is derived from human IgG₁. IgG1 is well known to bind to the proteins of the Fc-gamma receptor family (FcγR) as well as C1q. Interaction with these receptors can induce antibody-dependent cell cytotoxicity (ADCC) and complement-dependent cytotoxicity (CDC). Therefore, optionally, certain anti-Tim-3 antibodies described herein are a fully human monoclonal antibody lacking Fc effector function (IgG1, Fc-null). To achieve an Fc-null IgG1 antibody, selective mutagenesis of residues is necessary within the CH2 region of its IgG1 Fc region. Amino acid substitutions L234A, L235E, and G237A are introduced into IgG1 Fc to reduce binding to FcγRI, FcγRIIa, and FcγRIII, and substitutions A330S and P331S are introduced to reduce C1q-mediated complement fixation. To reduce the potential induction of an immune response when dosed in humans, certain amino acids may require back-mutations to match antibody germline sequences.

Optionally, certain anti-human PD-L1 antibodies described herein can contain an Fc portion which is derived from human IgG₁. IgG1 is well known to bind to the proteins of the Fc-gamma receptor family (FcγR) as well as C1q. Interaction with these receptors can induce antibody-dependent cell cytotoxicity (ADCC) and complement-dependent cytotoxicity (CDC). Therefore, optionally, certain anti-human PD-L1 antibodies described herein are a fully human monoclonal antibody lacking Fc effector function (IgG1, lambda, Fc-null). To achieve an Fc-null IgG1 antibody, selective mutagenesis of residues is necessary within the CH2 region of its IgG1 Fc region. Amino acid substitutions L234A, L235E, and G237A are introduced into IgG1 Fc to reduce binding to FcγRI, FcγRIIa, and FcγRIII, and substitutions A330S and P331S are introduced to reduce C1q-mediated complement fixation. To reduce the potential induction of an immune response when dosed in humans, certain amino acids may require back-mutations to match antibody germline sequences. As such, certain anti-human PD-L1 antibodies described herein contain E1Q and S94R mutations in the variable heavy chain, and contain T76S and A80S mutations in the variable light chain.

The HCVR and LCVR regions can be further subdivided into regions of hyper-variability, termed complementarity determining regions (“CDRs”), interspersed with regions that are more conserved, termed framework regions (“FR”). Each HCVR and LCVR is composed of three CDRs and four FRs, arranged from amino-terminus to carboxy-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues which form specific interactions with the antigen. There are currently three systems of CDR assignments for antibodies that are used for sequence delineation. The North CDR definition (North et al., “A New Clustering of Antibody CDR Loop Conformations”, Journal of Molecular Biology, 406, 228-256 (2011)) is based on affinity propagation clustering with a large number of crystal structures. For the purposes of the present invention, the North CDR definitions are used.

An isolated DNA encoding a HCVR region can be converted to a full-length heavy chain gene by operably linking the HCVR-encoding DNA to another DNA molecule encoding heavy chain constant regions. The sequences of human, as well as other mammalian, heavy chain constant region genes are known in the art. DNA fragments encompassing these regions can be obtained e.g., by standard PCR amplification.

An isolated DNA encoding a LCVR region may be converted to a full-length light chain gene by operably linking the LCVR-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a human kappa or lambda constant region. Preferably for anti-human Tim-3 antibodies of the present invention, the light chain constant region is a human kappa constant region.

The polynucleotides of the present invention will be expressed in a host cell after the sequences have been operably linked to an expression control sequence. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

The antibodies of the present invention may readily be produced in mammalian cells, non-limiting examples of which includes CHO, NS0, HEK293 or COS cells. The host cells are cultured using techniques well known in the art.

The vectors containing the polynucleotide sequences of interest (e.g., the polynucleotides encoding the polypeptides of the antibody and expression control sequences) can be transferred into the host cell by well-known methods, which vary depending on the type of cellular host.

Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994).

In other embodiments of the present invention, the antibody, or the nucleic acids encoding the same, is provided in isolated form. As used herein, the term “isolated” refers to a protein, peptide, or nucleic acid which is free or substantially free from any other macromolecular species found in a cellular environment. “Substantially free” as used herein means the protein, peptide, or nucleic acid of interest comprises more than 80% (on a molar basis) of the macromolecular species present, preferably more than 90%, and more preferably more than 95%.

The antibodies of the present invention, or pharmaceutical compositions comprising the same, may be administered by parenteral routes (e.g., subcutaneous and intravenous). The antibodies of the present invention may be administered to a patient along with pharmaceutically acceptable carriers, diluents, or excipients in single or multiple doses. Pharmaceutical compositions of the present invention can be prepared by methods well known in the art (e.g., Remington: The Science and Practice of Pharmacy, 22^{nd} ed. (2012), A. Loyd et al., Pharmaceutical Press) and comprise an antibody, as disclosed herein, and one or more pharmaceutically acceptable carriers, diluents, or excipients.

Dosage regimens may be adjusted to provide the optimum desired response (e.g., a therapeutic effect). Treatment dosages may be titrated to optimize safety and efficacy. Dosing schedules, for intravenous (i.v.) or non-intravenous administration, localized or systemic, or combinations, thereof will typically range from a single bolus dosage or continuous infusion to multiple administrations per day (e.g., every 4-6 hours), or as indicated by the treating physician and the patient's condition.

The term “treating” (or “treat” or “treatment”) refers to slowing, interrupting, arresting, alleviating, stopping, reducing, or reversing the progression or severity of an existing symptom, disorder, condition, or disease.

“Effective amount” means the amount of an antibody of the present invention or pharmaceutical composition comprising an antibody of the present invention that will elicit the biological or medical response of or desired therapeutic effect on a tissue, system, animal, mammal or human that is being sought by the researcher, medical doctor, or other clinician. An effective amount of the antibody may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects.

### Antibody Generation, Expression, and Purification

The antibodies of the present invention may be generated by known methods including, but not limited to, by using phage display, transgenic animals, and/or humanization. For generation of anti-human Tim-3 antibodies, the human Tim-3 protein can be pretreated with PNGaseF enzyme prior to use. Additionally, the antibodies derived as described above may be further screened using the assays described herein.

The polypeptides of the variable regions of the heavy chain and light chain, the complete heavy chain and light chain amino acid sequences for Antibodies A and B and the nucleotide sequences encoding the same, are listed in the section entitled “Amino Acid and Nucleotide Sequences.” In addition, the SEQ ID NOs for the light chain, heavy chain, light chain variable region, and heavy chain variable region of Antibodies A and B are also provided. The antibodies of the present invention, including, but not limited to, Antibodies A and B can be made and purified essentially as follows. An appropriate host cell, such as HEK 293 or CHO, can be either transiently or stably transfected with an expression system for secreting antibodies using an optimal predetermined HC:LC vector ratio or a single vector system encoding both HC (heavy chain) and LC (light chain). Clarified media, into which the antibody has been secreted, may be purified using any of many commonly-used techniques. For example, the medium may be conveniently applied to a Mab Select column (GE Healthcare), or KappaSelect column (GE Healthcare) for Fab fragment, that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7 to 10 mM sodium citrate buffer pH 3.0, or phosphate buffered saline pH 7.4 to 100 mM glycine buffer pH 3.0). Antibody fractions may be detected, such as by UV absorbance or SDS-PAGE, and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is typically greater than 95%. The product may be immediately frozen at −70° C. or may be lyophilized.

TABLE 1

| | Antibody | Antibody |
|---|---|---|
| | A (anti-Tim-3 | B (Anti-PD-L1 |
| Corresponding SEQ ID | Antibody) | Antibody) |
| HCDR1 | 2 | 17 |
| HCDR2 | 3 | 18 |
| HCDR3 | 4 | 19 |
| LCDR1 | 5 | 20 |
| LCDR2 | 6 | 21 |
| LCDR3 | 7 | 22 |
| HCVR | 8 | 23 |
| LCVR | 9 | 24 |
| Heavy chain | 10 | 25 |
| Light chain | 11 | 26 |
| DNA Heavy Chain | 12 | 27 |
| DNA Light Chain | 13 | 28 |

### WINN Assay

The antibodies of the present invention can be tested for in vivo immunomodulatory activity with the WINN assay. In the WINN assay, human NSCLC tumor cells NCI-H292 and human immune cells (allogeneic) are mixed and co-implanted into an immunodeficient mouse, and then followed by dosing with an immunomodulatory agent. The ability of the immunomodulatory agent to inhibit or delay tumor formation or support intra-tumroal persistence can be assessed as follows.

On day 0, NSG mice from Jackson Laboratories (7 weeks of age, female, in groups of 8-10 mice) are implanted into the flank subcutaneously with either 2×10⁶ H292 cells, or a mixture of 2×10⁶ H292 cells and 1×10⁶ human PBMCs in HBSS (0.2 ml total volume). Starting on Day 0, mice are treated with an i.p. injection of control human IgG at 10 mg/kg or Antibody A at 1 mg/kg or 10 mg/kg, one time per week for six weeks. Animal well-being and behavior, including grooming and ambulation, are monitored at least twice per week.

Tumor sections from the model can be analyzed for CD3-positive and CD8-positive T cell persistence by measuring the presence of CD3-positive and CD8-positive T cells by staining for CD3 and CD8 and analyzing with the Aperio ScanScope™. The IHC Nuclear Image Analysis macro detects nuclear staining for a target chromogen for the individual cells in those regions that are chosen by the user and quantifies their intensities. Three to five annotations are made from viable tumor area and used in adjusting the parameters until the algorithm results generate consistent cell identification. The macro is then saved and the slides logged in for analysis. The % CD3-positive and CD8-positive cells as a percent of the total number of cells are calculated by the Aperio software.

In experiments performed essentially as described in this WINN assay, by IHC analysis, mice co-implanted with NCI-H292 tumors and PBMCs and dosed with Antibody A at 10 mg/kg results in a significant increase (30%) of human CD3-positive CD8-positive intratumoral T cells as compared to mice co-implanted with NCI-H292 tumors and PBMCs and treated with the control IgG (6.5%) (P=0.03).

Established Human Tumor Xenograft Model in NSG Mice Humanized with Primary Human T Cells

The efficacy of the antibodies of the present invention can be tested in the NCI-HCC827 human NSCLC (non-small cell lung cancer) xenograft model to assess the ability to delay or destroy established tumors in the model. On day 0, 1×10⁷ NCI-HCC827 cells are implanted subcutaneously into the flank of NSG mice (7 weeks of age, female, 8 mice per group). When tumors reach a volume of ˜400 mm³ (˜days 30-32), the mice are infused (i.v.) with 2.5×10⁶ previously expanded human T cells. Previously expanded human T cells are generated by isolating human T cells from whole blood and expanding using Dynabeads® Human T-Activator CD3/CD28 for 10 days. Previously expanded human T cells may be cryopreserved for later use. One day after T cell infusion, mice are dosed at 10 mg/kg by weekly (4 total doses) i.p. injection with human IgG or Antibody A. Animal well-being and behavior, including grooming and ambulation are monitored at least twice per week.

Body weight and tumor volume are measured twice a week. Tumor volumes were measured twice per week starting on day 4 post-cell implantation using electronic calipers as described above. Tumor Volume (mm³)=π/6*Length*Width². The antitumor efficacy is expressed as T/C ratio in percent and calculated as summarized below: % T/C is calculated by the formula 100 ΔT/ΔC if ΔT>0 of the geometric mean values. ΔT=mean tumor volume of the drug-treated group on the final day of the study−mean tumor volume of the drug-treated group on initial day of dosing; ΔC=mean tumor volume of the control group on the final day of the study−mean tumor volume of the control group on initial day of dosing. Additionally, % Regression is calculated using the formula=100×ΔT/Tᵢₙᵢₜᵢₐₗ if ΔT<0. Animals with no measurable tumors are considered as Complete Responders (CR) and tumors with >50% regressions are Partial Responders (PR).

In experiments performed essentially as described above, treatment with Antibody A (anti-human Tim-3) significantly inhibits tumor growth in the humanized NSG mice, compared to treatment with human IgG (Table 2). On day 76, treatment with Antibody A results in a T/C=2%. On day 110, Antibody A treatment results in a ⅜ CR.

TABLE 2
Tumor volume (mm³) in the NCI-HCC827 human
NSCLC xenograft model

| | |
|---|---|
| Human IgG | |
| Control | Antibody A |

| Day | Mean | SEM | Mean | SEM |
|---|---|---|---|---|
| 21 | 152 | 13 | 164 | 85 |
| 28 | 289 | 24 | 309 | 160 |
| 30 | 332 | 28 | 358 | 186 |
| 34 | 388 | 32 | 403 | 209 |
| 36 | 414 | 34 | 505 | 262 |
| 40 | 706 | 59 | 628 | 326 |
| 43 | 752 | 62 | 733 | 380 |
| 47 | 858 | 71 | 763 | 396 |
| 50 | 932 | 77 | 747 | 387 |
| 55 | 982 | 81 | 807 | 418 |
| 57 | 1212 | 100 | 843 | 437 |
| 62 | 1324 | 110 | 553 | 287 |
| 65 | 1524 | 126 | 726 | 376 |
| 69 | 1492 | 124 | 602 | 312 |
| 72 | 1827 | 151 | 539 | 279 |
| 76 | 2030 | 168 | 375 | 196 |
| 79 | | | 375 | 196 |
| 83 | | | 414 | 218 |
| 85 | | | 331 | 175 |
| 90 | | | 192 | 103 |
| 93 | | | 235 | 128 |
| 97 | | | 173 | 95 |
| 100 | | | 118 | 65 |
| 103 | | | 125 | 69 |
| 106 | | | 120 | 67 |
| 110 | | | 131 | 73 |

### Mixed Lymphocyte Reaction

The function of blocking Tim-3 signals by antibodies of the present invention may be evaluated by measuring the release of cytokines during T cell activation. The levels of certain cytokines, such as IFN-γ, are expected to increase if T cell activation is promoted by treatment with antibodies of the present invention.

CD14⁺ monocytes are isolated by negative selection from fresh human PBMC obtained from a healthy donor (AllCells) using human monocyte isolation kit II (Miltennyi Biotec). Human monocyte-derived dendritic cells are generated by culturing the CD14⁺ monocytes in complete RPMI-1640 medium in the presence of 62.5 ng/ml hGM-CSF and 20 ng/ml hIL-4 for 7 days. CD4⁺ T cells are purified from fresh human PBMC of a different healthy donor (AllCells) by negative selection using the CD4 T cell isolation kit (Miltenyi). The two types of cells are then mixed in individual wells of a 96-well plate with 100 μl complete AIM-V medium containing 1×10⁵ CD4⁺ T cells and 2×10⁴ immature DC per well. 100 μl complete AIM-V medium is added containing 100 nM human IgG1 or Antibody A in 6 replicates. After incubation for 3 days at 37° C. at 5% CO₂, supernatants are harvested and measured for human IFN-γ with an ELISA kit (R&D Systems). An unpaired t-test is used to compare groups.

In experiments performed essentially as described above, the addition of Antibody A significantly increases the secretion of IFN-γ as compared to the addition of control human IgG1 (3,036±367 vs. 1,644±261 pg/mL of hIFN-γ; p=0.0384).

### ELISA Analysis: Antibody a Binds to Recombinant Tim-3

The ability of antibodies of the present invention to bind human Tim-3 can be measured with an ELISA assay. For the Tim-3 binding assay, a 96-well plate (Nunc) is coated with human Tim-3-Fc (R&D Systems) overnight at 4° C. Wells are blocked for 2 h with blocking buffer (PBS containing 3% bovine serum albumin). Wells are washed three times with PBS containing 0.1% Tween-20. Antibody A or control IgG (100 μl) is then added and incubated at room temperature for 1 h. After washing, the plate is incubated with 100 μl of goat anti-human IgG F(ab′)2-HRP conjugate (Jackson Immuno Research) at room temperature for 1 h. The plates are washed and then incubated with 100 μl of 3,3′,5,5′-tetra-methylbenzidine. The absorbance at 450 nm is read on a microplate reader. The half maximal effective concentration (EC50) is calculated using GraphPad Prism 6 software.

In experiments performed essentially as described above, Antibody A binds human Tim-3 with an EC50 of 2.07×10⁻¹¹ M.

### Flow Cytometric Analysis: Antibody a Binds to Cell Surface Tim-3

The ability for antibodies of the present invention to bind to cell surface human Tim-3 can be measured with a flow cytometric assay. Tim-3 DO11.10 cells, a human Tim-3 expressing DO11.10 cell line, are used for this assay.

Tim-3 DO11.10 cells can be obtained as follows. Full-length Tim-3 gene can be purchased from Origene Technologies, Inc. and cloned into a pLVX-IRES-Neo lentivirus vector from Clonetech Laboraties, Inc. using PCR. Lenti-X™ system from Clonetech Laboraties, Inc. is used to generate high titers of recombinant, replication-incompetent virions. The virions are either used to infect the target cells immediately or are aliquoted and frozen at −80 until use. The murine T cell hybridoma, DO11.10 cell line, can be obtained from the National Jewish Health®. The DO11.10 cells are cultured and maintained according to a protocol accompanying this cell line. On day 0, DO11.10 cells are counted and spun down to remove culture media. Cell pellets are mixed with virions containing the human TIM-3 gene or vector control and incubated at 37° C. for 24 hours. Polybrene is added when mixing cells and virions until a final concentration of 8 ug/ml is achieved. After 24 hours, DO11.10 cells are pelleted again and resuspended in fresh culture media and incubated at 37° C. for 3 days. Next, the DO11.10 cells are pelleted every 3 days and resuspended in selection media containing 1 mg/ml Geneticin® to select stably transduced cells. Tim-3 expression is monitored by flow cytometry using antibodies obtained from R&D Systems. After 2 to 3 weeks in selection media, the resulting Tim-3 expressing DO11.10 cells are sorted to establish a single cell clone.

DO11.10 and Tim-3 DO11.10 cells are added to a 96 well V-bottom plate at 1.×10⁵ cells per well (100 μl/well) in staining buffer (DPBS containing 3% BSA). Cells are Fc blocked on ice for 1 hour in staining buffer with 30 μg/mL human IgG. Antibody A or control human IgG is labelled with A488 (Molecular Probes®) and 12 point titrations (1:3 serial dilutions) of both antibodies are prepared in staining buffer with a starting concentration of 66.7 nM. Labelled antibodies are added to the cells and incubated for 1 hour at 4° C. in the dark. Cells are washed two times with PBS by spinning for 5 min at 1200 RPM and decanting the supernatant. Live/Dead cell dye 7-AAD (1:1000 in PBS) is added to each well at 3 μl/well and cells are incubated for 15 min on ice. Cells are washed two times with PBS and resuspended in 100 μl DPBS containing 0.5% BSA and analyzed on an Intellictye iQue. All stainings are done in triplicate. Data are analyzed with FlowJo software to identify populations of live cells and determine the median fluorescence intensity of each sample using the AF488 (FL1) detection channel. The individual MFI (i.e. mean fluorescence intensity) values are placed into GraphPad Prism software to generate concentration response curves from which EC50 values are extrapolated.

In experiments performed essentially as described above, Antibody A binds to cellular bound human Tim-3 on Tim-3 DO11.10 cells in a dose dependent manner with an EC50 value of 0.09 nM.

Flow Cytometric Analysis: Antibody a Blocks the Interaction of Phosphatidylserine with Human Tim-3

The ability for certain antibodies of the present invention to block phosphatidylserine binding to Tim-3 can be measured by FACS analysis. For this receptor-ligand blocking assay, 1×10⁶/ml of DO11.10 cells are treated with 12 μM camptothecin (Sigma®) for 3 hours at 37° C. to induce apoptosis. FITC-Annexin V (Becton Dickinson®) is used as a positive control to detect the existence of phosphatidylserine. Biotinylated hTIM-3-Fc binds strongly to camptothecin-treated cells but does not bind to non-treated cells. Camptothecin-treated cells are washed with cold PBS and resuspended in binding buffer (Becton Dicknson®) at 1×10⁶ cells/ml. Fc receptors are blocked by adding 50 μg/ml mouse IgG and rat IgG to the cells and incubating at room temperature for 30 min. 6 point titrations (1:3 serial dilutions) of Antibody A are prepared in binding buffer with a starting concentration of 90 nM and added to 1 ml of cells and cells are then incubated for 60 min at room temperature. hTIM-3-Fc Biotin is then added at 0.05 μg/well to the appropriate samples in a 200 μl volume and incubated for 30 min at room temperature. Cells are then washed twice with binding buffer by centrifugation at 1200 RPM for 5 min. 2.4 μl/well of a streptavidin-FITC (Biolegend®) containing solution (1:10 dilution in DPBS) and 5 μl/well of propidium iodide are added to each well and incubated for 30 min at room temperature in the dark. Cells are washed twice with binding buffer and resuspended in 100 μl of PBS. Samples are read on the IntelliCyt iQue Flow Cytometer and Data were analyzed with FlowJo software. The individual MFI (i.e. mean fluorescence intensity) values are placed into GraphPad Prism software to generate concentration response curves from which IC50 values are extrapolated.

In experiments performed essentially as described above, Antibody A blocks the interaction of human Tim-3 with phosphatidylserine in a dose-dependent manner with an IC50 value of 0.32 nM and as further illustrated in Table 3.

TABLE 3

| | Untreated DO11.10 + | | | | | | | |
|---|---|---|---|---|---|---|---|---|
| | hTIM-3-Fc Biotin | Camptothecin-treated DO11.10 + hTIM-3-Fc Biotin | | | | | | |
| | Antibody A (nM) | | | | | | | |
| | 0 | 90 | 30 | 10 | 3.3 | 1.1 | 0.37 | 0 |
| MFI | 1747 | 1815 | 19655 | 32574 | 52885 | 96566 | 197146 | 214044 |

Galectin-9 Blocking Assay: Antibody a Blocks the Interaction of Human Galectin-9 with Human Tim-3

The ability for antibodies of the present invention to block human galectin-9 binding to human Tim-3 can be measured as follows. For this receptor-ligand blocking assay, a 96-well streptavidin-coated MSD plate (Meso Scale Diagnostics) is blocked for 2 hours with 150 μl blocking buffer (PBST containing 5% bovine serum albumin). Wells are washed three times with 200 μl PBS containing 0.2% Tween-20. Recombinant human galectin-9 (R&D Systems) is biotinylated using EZ-Link™ biotin (Thermo Scientific™) and then 25 μl of 0.21 μg/ml of the human recombinant galectin-9-biotin is then added and incubated at room temperature for 2 hours. Plates are washed three times with PBS containing 0.2% Tween-20. Human Tim-3-Fc protein (R&D Systems) is ruthinylated using sulfo-tag NETS-ester reagent (Meso Scale Discovery®) and a small aliquot is stored at −80 until use. Antibodies are serially diluted (starting at 13.5 μg/ml) and 50 μl of each antibody combined with 50 μl of diluted hTim-3-Fc-ruth at 0.05 μg/ml and incubated for 1 hour at room temperature. 50 μl of each combination is then added to the plate and incubated for 1.5 hours at room temperature. Plates are washed three times with PBS containing 0.2% Tween-20. 150 μl of 1× read buffer (Meso Scale Diagnostics) is then added to each well of the plate and the plate is read on a Sector Imager 2400 (Meso Scale Diagnostics).

In experiments performed essentially as described above, Antibody A blocks the interaction of human Tim-3 with human galectin-9 with an IC50 value of 5.6 nM as compared to control a polyclonal anti-human Tim-3 antibody (R&D Systems) with an IC50 value of 7.8 nM. However, The polyclonal anti-human Tim-3 antibody can block up to 100% human Tim-3's interactions with human galectin-9 while Antibody A only achieve partial blockage in this assay.

CEACAM-1 Blocking Assay: Antibody a does not Block the Interaction of Human CEACAM1 with Human Tim-3

The ability for antibodies of the present invention to block human CEACAM1 binding to human Tim-3 can be measured as follows. For this receptor-ligand blocking assay, a 96-well Immulon 4HBX plate (Thermo Scientific) is coated with 100 μl/well of 1 ug/ml human Tim-3-Fc at 4° C. The plate is washed three times with PBS containing 0.2% Tween-20 and blocked with 200 μl/well of PBS with 3% BSA for 1 hour at room temperature. Blocking buffer is then removed and 50 μl of titrated Abs (including polyclonal anti-human Tim-3, R&D Systems, Antibody A, and control human IgG), starting at 600 nM are added to the plate and incubated for 1 hour at room temperature. 50 μl of 20 μg/ml of CEACAM1 (BIOTANG) is then added directly to the wells and incubated for 1 hour at room temperature (final concentration of antibody is 300 nM and of CEACAM1 is 10 μg/ml). The plate is washed three times with PBS containing 0.2% Tween-20 and 100 μl of 0.2 μg/ml of biotinylated human CEACAM1 antibody (R&D Systems) is added and then incubated for 1 hour at room temperature. The plate is washed three times with PBS containing 0.2% Tween-20 and then 100 μl of streptavidin peroxidase (Jackson ImmunoResearch Laboratories) is added and then incubated for 1 hour at room temperature. The plate is washed six times with PBS containing 0.2% Tween-20 and developed using 100 μl/well of a 1:1 TMB substrate solution A and B (KPL) for 10 min at room temperature. The reaction is then stopped with 100 μl/well of 0.1N 142504 and the plate is read on a SpectraMax® plate reader at 450 nm.

In experiments performed essentially as described above, Antibody A does not significantly block the binding of CEACAM1 to human Tim-3, as illustrated in Table 4 below.

TABLE 4

| | Concentration of Antibody (nM) | | | | | | | | | |
|---|---|---|---|---|---|---|---|---|---|---|
| | 0.015 | 0.046 | 0.137 | 0.41 | 1.24 | 3.71 | 11.1 | 33.3 | 100 | 300 |
| Human | 2.05 | 2.02 | 2.13 | 2.03 | 2.04 | 2.03 | 2.05 | 2.07 | 2.12 | 2.08 |
| IgG Control | | | | | | | | | | |
| (O.D.) | | | | | | | | | | |
| Polyclonal | 1.96 | 1.88 | 1.89 | 1.88 | 1.85 | 1.80 | 1.51 | 1.16 | 0.99 | 0.99 |
| Anti-Tim-3 | | | | | | | | | | |
| (O.D.) | | | | | | | | | | |
| Antibody A | 1.87 | 1.88 | 1.87 | 1.82 | 1.80 | 1.78 | 1.79 | 1.79 | 1.73 | 1.74 |
| (O.D.) | | | | | | | | | | |

### Epitope

A Fab for Antibody A is generated by by enzymatically clipping Antibody A with immobilized (agarose resin) papain (ThermoFisher Scientific) followed by a standard ProA column (GE Healthcare Life Sciences) purification to pull out the free, soluble Fc and the unclipped IgG. Flow through containing the Fab is collected to concentrate and buffer exchange. The hTim-3-IgV-FLAG is purified from the 293HEK supernatant with a standard anti-FLAG resin (Sigma-Aldrich) protocol. The hTim-3-IgV domain represents amino acid residues S22 to K130 of human Tim-3 (SEQ ID:1). Flow through is rerun in the resin column multiple times. After each run, SDS-PAGE (NuPAGE Novex 4-12% Bis-Tris Gels; Invitrogen) and HPLC (TSKgel G3000 SW XL (Dimensions: 7.8 mm, ID 30 CM, 5 μM; TOSHO BioSCIENCE) is utilized to determine quality of the hTim-3-FLAG protein. Proteins of the best rounds are combined together to generate the final batch.

hTim-3-IgV-FLAG, at 2.17 mg/mL in TBS buffer pH 7.2, and Antibody A-Fab, at 6.79 mg/mL, are combined in a 1:1 molar ratio and the complex is isolated via size exclusion chromatography with a final concentration of 6.9 mg/mL in 20 mM hepes pH 7.4 and 150 mM sodium chloride. The Tim-3-anti-Tim-3 complex is screened in five Qiagen grid screens at both 8° C. and 21° C. using the sitting drop vapor diffusion method. Drops are set up using an Art Robbins Phoenix liquid handling robot which dispenses 0.3 μL crystallization solution on top of 0.3 μL protein. 100-200 μm intergrown prisms are obtained at 21° C. in 20% PEG 3350 and 0.2 M lithium chloride. Crystals are harvested and cryoprotected in a solution made of the crystallization condition supplemented with 20% ethylene glycol prior to flash freezing in liquid nitrogen. A dataset is collected at Argonne National Laboratory diffracting to 2.2 Å in space group P21 with cell parameters a=74.62 Å, b=57.85 Å, and c=74.71 Å.

The structure of the Antibody A-Fab in complex with human Tim-3 is determined by Molecular Replacement using the program Phaser. High resolution and publicly available Fab structures and the published structure of murine Tim-3 can be used as Molecular Replacement models. The structure is refined using the program Refmac and the model rebuilt using the program COOT. Final refinement R-factors are Rwork=20.2%, Rfree=23.4%. There are no Ramachandran violators, and 96.4% of the residues are in the favored region of the Ramachandran plot. There is density indicating glycosylation at Asn99 of Tim-3 (SEQ ID NO:1).

Biacore T200 is utilized to determine the binding kinetics of hTim-3-IgV-FLAG to the captured AntibodyA-Fab. In HBS-EP as a running buffer, 1:1 binding of this complex at 25° C. has a kₒₙ of 3.62E+05 1/Ms, k_{off} of 2.86E-03 1/s, and a K_{D} of 7.92E-09 M.

In experiments performed essentially as described in this assay, Antibody A-Fab/hTim-3 complex is resolved and the epitope/paratope is illustrated in Table 5 below. Table 5 below lists the residues on Antibody A-Fab that are within 6A of the listed residues on hTim-3 (SEQ ID NO:1). The heavy chain of the Antibody A-Fab has 62 contacts (cutoff 6 Å) with hTim-3 while the light chain has 34 contacts (cutoff 6 Å).

TABLE 5

| | Antibody A | Antibody A |
|---|---|---|
| Tim-3 | Heavy Chain | Light Chain |
| (Epitope) | (Paratope) | (Paratope) |
| P50 | S54 | — |
| K55 | — | Y32 |
| G56 | — | Y32 |
| A57 | — | Y30, Y32, N92 |
| C58 | — | Y32, A91, N92, |
| | | S93 |
| P59 | Y99, T102 | Y32, A91, N92, |
| | | S93 |
| V60 | Y59, Y99, T102 | Y32, Q89, |
| | | Q90, A91, N92, |
| | | S93, F94, P95, P96 |
| F61 | Y33, S35, W47, | A91, F94, P96 |
| | A50, Y59, Y99, | |
| | A100, T102, F104 | |
| E62 | S31, Y33, Y59, | — |
| | Y99, R101 | |
| C63 | Y99, R101, T102 | Y32 |
| G64 | T102 | Y32 |
| N65 | T102 | N31, Y32, A50 |
| E72 | S54 | — |
| I107 | — | T30 |
| R111 | Y33, Y59 | — |
| Q113 | Y33, S52, G53, | — |
| | S54, G55, G56, | |
| | S57,Y59 | |
| I114 | G56, S57 | — |
| P115 | G56, S57 | — |
| G116 | G56, S57, T58, | — |
| | Y59 | |
| I117 | G56, S57, T58, | — |
| | Y59, Y60, K65 | |
| M118 | S57, T58, Y59, | F94 |
| | Y60, A61, D62, | |
| | K65 | |
| N119 | T58, Y59 | — |
| D120 | Y33, S57, Y59 | — |
| K122 | — | N92, F94 |

### Kinetics/Affinity Study for Antibody A

A Biacore T100 instrument can be used to measure the kinetics of human Tim-3-IgV-Fc single arm antigen (SAG) binding to captured Antibody A. Human Fab Binder surfaces are prepared by amine-coupling Human Fab Binder (GE Healthcare) to a Biacore CM5 sensor chip surface. Test antibodies are captured by the chip using HBS-EP buffer (GE Healthcare) as the running buffer. Tim-3 SAG is diluted into running buffer starting at 30 nM with a dilution factor of 3 to give concentrations of 0.04, 0.12, 0.37, 1.11, 3.33, 10 and 30 nM. Diluted Tim-3 SAG analyte or buffer is injected at 30 μl/min for 180 seconds and the complex dissociation is monitored for 1200 seconds. The binding surface is regenerated with injection of 10 mM Glycine-HCl pH 2.1 at 30 μl/min, 30 seconds of two injections for five lower concentrations, and two injections at 60 seconds for two higher concentrations between each analyte binding cycle. Experimental data for a given antigen/Ab interaction are fit using a 1:1 Langmuir with mass transport Model.

In experiments performed essentially as described above, Antibody A binds to human Tim-3 with the kinetics and affinity constants illustrated in Table 6.

TABLE 6

| Antibody | Kₒₙ (1/Ms) | K_{off} (1/s) | K_{D} (M) | Rₘₐₓ | Chi² |
|---|---|---|---|---|---|
| Antibody A | 2.33E+06 | 9.27E−04 | 3.98E−10 | 17.09 | 0.319 |

Established Human Tumor Xenograft Model in NSG Mice Humanized with PBMC and Antibody B

The efficacy of Antibody B can be tested in the NCI-H827 human NSCLC xenograft model to assess the ability to delay or destroy established tumors in the model. On day 0, 1×10⁷ H827 cells are implanted subcutaneously into the flank of NSG mice (7 weeks of age, female, 10 mice per group). With the human xenograft tumor established, the mice are infused (i.v.) with 5×10⁶ human PBMCs on day 34. Starting on day 35, mice are dosed at 10 mg/kg by weekly (3 total doses) i.p. with either human IgG or Antibody B (anti-PD-L1 antibody). Animal well-being and behavior, including grooming and ambulation are monitored at least twice per week. Body weight and tumor volume are measured twice a week.

In experiments performed essentially as described in this assay, treatment with Antibody B significantly inhibits tumor growth in the humanized NSG mice, compared to treatment with human IgG (Table 7).

TABLE 7
Tumor volume (mm³) in the NCI-H827 human NSCLC xenograft model

| Treatment | Days | 21 | 28 | 30 | 34 | 36 | 40 | 43 | 47 |
|---|---|---|---|---|---|---|---|---|---|
| Hu IgG | Mean | 156 | 290 | 337 | 397 | 445 | 726 | 779 | 883 |
| | SEM | 15 | 13 | 24 | 34 | 60 | 59 | 75 | 78 |
| Antibody B | Mean | 163 | 293 | 336 | 367 | 379 | 433 | 557 | 468 |
| | SEM | 13 | 26 | 25 | 20 | 51 | 35 | 54 | 41 |
| Treatment | Days | 50 | 55 | 57 | 62 | 65 | 69 | 72 | 76 |
| Hu IgG | Mean | 959 | 1000 | 1241 | 1345 | 1530 | 1508 | 1854 | 2056 |
| | SEM | 87 | 69 | 102 | 91 | 52 | 90 | 121 | 123 |
| Antibody B | Mean | 503 | 593 | 580 | 672 | 625 | 775 | 772 | 691 |
| | SEM | 76 | 85 | 105 | 154 | 170 | 202 | 221 | 231 |

### Binding Kinetics and Affinity

The kinetics and equilibrium dissociation constant (K_{D}) for human PD-L1 is determined for Antibody B using surface plasmon resonance (Biacore).

Immobilization of Antibody B as ligand on to sensor chip surface is performed at 25° C. Soluble human PD-L1-Fc fusion protein (and in some cases, cynomolgus monkey PD-L1-Fc fusion proteins) is injected as analyte at concentrations ranging from 0.0123 nM-9 nM. The analysis is performed at 37° C. The contact time for each sample is 180 sec at 30 μl/min. The dissociation time was 240-1500 seconds. The immobilized surface is regenerated for 18 seconds with 0.95 M NaCl/25 mM NaOH at 30 μl/min, and then stabilized for 30 seconds. Binding kinetics are analyzed using the Biacore T200 Evaluation software (Version 3.0). Data are referenced to a blank flow cell, and the data are fit to a 1:1 binding model.

In experiments performed essentially as described in this assay, Antibody B binds to human PD-L1 with a K_{D} of 82 pM.

TABLE 8
Binding by SPR of Antibody B

| Binding to Antibody B | Kon (1/Ms) | Koff (1/s) | K_{D} (pM) |
|---|---|---|---|
| Human PD-L1 | 1.40E+06 | 1.14E−04 | 82 |
| Cyno PD-L1 | 1.51E+06 | 1.84E−04 | 122 |

### ELISA Analysis: Antibody B Binds to Recombinant PD-L1

The ability of Antibody B to bind human PD-L1 can be measured by ELISA. For the PD-L1 binding assay, a 96-well plate (Nunc) is coated with human PD-L1-Fc (R&D Systems) overnight at 4° C. Wells are blocked for 2 h with blocking buffer (PBS containing 5% nonfat dry milk). Wells are washed three times with PBS containing 0.1% Tween-20. Antibody B or control IgG (100 ul) is then added and incubated at room temperature for 1 h. After washing, the plate is incubated with 100 μl of goat anti-human IgG F(ab′)2-HRP conjugate (Jackson Immuno Research) at room temperature for 1 h. The plates are washed and then incubated with 100 μl of 3,3′,5,5′-tetra-methylbenzidine. The absorbance at 450 nm is read on a microplate reader. The half maximal effective concentration (EC50) is calculated using GraphPad Prism 6 software.

In experiments performed essentially as described in this assay, Antibody B binds to human PD-L1 with an EC50 of 0.11 nM. Antibody B retains its binding activities after 4 weeks under all three temperature conditions, 4° C., 25° C. and 40° C.

### Flow Cytometric Analysis: Antibody B Binds to Cell Surface PD-L1

The ability of Antibody B to bind to cell surface expressed human PD-L1 can be measured by flow cytometry. MDA-MB 231 cells (a PD-L1-positive human breast adenocarcinoma cell line) are added to a 96 well U-bottom plate at 1.5×10⁵ cells per well in 200 μl staining buffer and incubated at 4° C. for 30 min. Plates are centrifuged at 1200 rpm for 5 min and supernatant removed. 100 μl of Antibody B-biotin (serially diluted by 1:4 starting from 10 ug/ml) is added. A total of 6 serial dilutions are evaluated. After incubation at 4° C. for 30 min, cells are washed twice with DPBS. 100 μl of detection buffer containing 5 μl streptavidin-PE is added. After incubation at 4° C. for 30 more min, plate is centrifuged and washed twice with DPBS. Cells are re-suspended in 200 μl DPBS for FACS analysis.

In experiments performed essentially as described in this assay, Antibody B binds to cell surface PD-L1 on MDA-MB231 cells in a dose dependent manner with an EC50 of 0.14 nM.

ELISA Analysis: Antibody B Blocks the Interaction of PD-L1 with PD-1

The ability for antibodies of the present invention to block PD-L1 binding to PD-1 can be measured by ELISA. For the receptor-ligand blocking assay, varying amounts of Antibody B or control IgG are mixed with a fixed amount of biotinylated PD-L1-Fc fusion protein (100 ng/well) and incubated at room temperature for 1 h. The mixture is transferred to 96-well plates pre-coated with PD-1-Fc (1 μg/ml) and then incubated at room temperature for an additional 1 h. After washing, streptavidin HRP conjugate is added, and the absorbance at 450 nm is read. IC50 represents the antibody concentration required for 50% inhibition of PD-L1 binding to PD-1.

In experiments performed essentially as described in this assay, Antibody B blocks the interaction of PD-L1 with PD-1 with an IC50 of 0.95 nM. Antibody B retains its blocking activities after 4 weeks under all three temperature conditions, 4° C., 25° C. and 40° C.

ELISA Analysis: Antibody B Blocks the Interaction of PD-L1 with B7-1

Human PD-L1 also binds to B7-1. The ability of Antibody B to block PD-L1 binding to B7-1 can be measured by ELISA. The procedure for the PD-L1/B7-1 blocking assay is similar to the PD-L1/PD-1 blocking assay, except that the plates are coated with 1 μg/ml B7-1-Fc (R&D Systems). The antibody concentration required for 50% inhibition of PD-L1 binding to PD-1 (IC50) is calculated using GraphPad prism 6 software.

In experiments performed essentially as described in this assay, Antibody B blocks the interaction of PD-L1 with B7-1 with an IC50 of 2.4 nM.

Antibody a Enhances Interferon-Gamma Production from In Vitro Stimulated Human PBMCs in the Presence of an Anti-Human PD-L1 Antibody

The function of blocking Tim-3 signals by antibodies of the present invention may be evaluated by measuring the release of cytokines during T cell activation. The levels of certain cytokines, such as IFN-γ, are expected to increase if T cell activation is promoted by treatment with antibodies of the present invention.

CD14⁺ monocytes are isolated by negative selection from fresh human PBMC obtained from a healthy donor (AllCells) using human monocyte isolation kit II (Miltennyi Biotec). Human monocyte-derived dendritic cells are generated by culturing the CD14⁺ monocytes in complete RPMI-1640 medium in the presence of 62.5 ng/ml hGM-CSF and 20 ng/ml human IL-4 for 3 days. Fresh human PBMC were isolated from different healthy donor (AllCells). The two types of cells are then mixed in individual wells of a 96-well plate with 100 μl complete AIM-V medium containing 7.5×10⁴ PBMC cells and 1.5×10⁴ immature DC per well. 100 μl complete AIM-V medium is added containing 100 nM human IgG1, 100 nM Antibody A, 4 nM or 1.33 nM atezolizumab, 0.07 nM or 0.22 nM Lilly PD-L1 antibody Antibody B, or 100 nM Antibody A in combination with atezolizumab or Antibody B in 8 replicates. After incubation for 6 days at 37° C. at 5% CO₂, supernatants are harvested and measured for human IFN-γ with an ELISA kit (R&D Systems). An unpaired t-test is used to compare groups.

In experiments performed essentially as described above, the addition of Antibody A or atezolizumab increases the secretion of IFN-γ as compared to the addition of human IgG1. The combination of Antibody A with atezolizumab significantly increases the secretion of IFN-γ as compared to the addition of atezolizumab alone at the dose of 1.33 nM dose (P=0.0014), as illustrated in Table 9 below. Antibody A provides an increase in the secretion of IFN-γ when in combination with Antibody B in this MLR assay (Table 10).

TABLE 9
Antibody A in combination with Atezolizumab results in an increase in T cell IFN-gamma production

| | Control IgG (100 nM)+ | | | Antibody A (100 nM)+ | | |
|---|---|---|---|---|---|---|
| Atezolizumab | 0 nM | 1.33 nM | 4 nM | 0 nM | 1.33 nM | 4 nM |
| IFN-gamma | 1427.48 ± 325.3 | 2523.6 ± 278.8 | 3383.42 ± 421.04 | 1494.13 ± 248.03 | 3463.11 ± 434.43 | 3129.87 ± 782.92 |
| (pg/ml) | | | | | | |

TABLE 10
Antibody A in combination with Antibody B results in an increase in T cell IFN-gamma secretion

| | Control | | Antibody B | | Antibody B | |
|---|---|---|---|---|---|---|
| | IgG (100 nM) | — | 0.07 nM | 0.22 nM | 0.07 nM | 0.22 nM |
| | Antibody A | | | | | |
| | — | 100 nM | — | — | 100 nM | 100 nM |
| IFN- gamma | 964.236 ± 112 | 1175.03 ± 100 | 1816.41 ± 301.1 | 2281.64 ± 183.8 | 2409.24 ± 453.5 | 2966.53 ± 269.9 |
| (pg/ml) | | | | | | |

Established Human Tumor Xenograft Model in NSG Mice Humanized with Primary Human T Cells

The efficacy of the antibodies of the present invention can be tested in the NCI-HCC827 human NSCLC (non-small cell lung cancer) xenograft model to assess the ability to delay or destroy established tumors in the model. On day 0, 1×10⁷ NCI-HCC827 cells are implanted subcutaneously into the flank of NSG mice (7 weeks of age, female, 8 mice per group). When tumors reach a volume of ˜400 mm³ (˜days 30-32), the mice are infused (i.v.) with 2.5×10⁶ previously expanded human T cells. Previously expanded human T cells are generated by isolating human T cells from whole blood and expanding using Dynabeads® Human T-Activator CD3/CD28 for 10 days. Previously expanded human T cells may be cryopreserved for later use. Same day after T cell infusion, mice are dosed at 10 mg/kg by weekly (4 total doses) i.p. injection with human IgG or Antibody A. Animal well-being and behavior, including grooming and ambulation are monitored at least twice per week.

Body weight and tumor volume are measured twice a week. Tumor volumes were measured twice per week starting on day 4 post-cell implantation using electronic calipers as described above. Tumor Volume (mm³)=π/6*Length*Width². The antitumor efficacy is expressed as T/C ratio in percent and calculated as summarized below: % T/C is calculated by the formula 100 ΔT/ΔC if ΔT>0 of the geometric mean values. ΔT=mean tumor volume of the drug-treated group on the final day of the study−mean tumor volume of the drug-treated group on initial day of dosing; ΔC=mean tumor volume of the control group on the final day of the study−mean tumor volume of the control group on initial day of dosing. Additionally, % Regression is calculated using the formula 100×ΔT/Tᵢₙᵢₜᵢₐₗ if ΔT<0. Animals with no measurable tumors are considered as Complete Responders (CR) and tumors with >50% regressions are Partial Responders (PR).

In experiments performed essentially as described above, all 3 treatments showed tumor growth inhibition post 4 weekly dosing through day 63, followed by tumor regrowth after the cessation of treatment (Table 11). Antibody A treatment at 10 mg/kg delays tumor growth with a T/C % of 57% through day 63 (p=0.065). Antibody B significantly inhibited the growth of HCC827 tumors with a T/C % of 29% through day 63, followed by tumor regrowth. The combination of Antibody A and Antibody B did not offer additional anti-tumor benefits versus Antibody B treatment alone (p=0.84) in this model.

TABLE 11
Tumor volume (mm³) in the NCI-HCC827 human NSCLC xenograft model

| | Human IgG | | | | | | Antibody B + | |
|---|---|---|---|---|---|---|---|---|
| | Control | | Antibody B | | Antibody A | | Antibody A | |
| Day | Mean | SEM | Mean | SEM | Mean | SEM | Mean | SEM |
| 15 | 106.22 | 8.16 | 105.04 | 8.04 | 106.91 | 7.48 | 106.50 | 5.44 |
| 24 | 189.95 | 10.05 | 182.32 | 13.31 | 194.56 | 8.01 | 195.97 | 9.05 |
| 27 | 230.81 | 4.60 | 247.85 | 10.65 | 257.03 | 7.84 | 239.26 | 16.06 |
| 30 | 287.36 | 3.87 | 281.06 | 8.19 | 276.72 | 8.35 | 294.67 | 8.64 |
| 35 | 415.25 | 27.08 | 410.49 | 60.70 | 416.23 | 17.33 | 409.17 | 48.26 |
| 38 | 523.76 | 34.15 | 493.53 | 72.98 | 516.64 | 21.51 | 496.85 | 58.60 |
| 42 | 559.33 | 36.48 | 455.37 | 67.34 | 628.06 | 26.15 | 481.89 | 56.84 |
| 45 | 702.14 | 45.79 | 469.50 | 69.43 | 618.22 | 25.74 | 513.69 | 60.59 |
| 49 | 762.12 | 49.70 | 561.92 | 83.09 | 762.93 | 31.77 | 622.72 | 73.45 |
| 52 | 795.47 | 51.88 | 521.83 | 77.17 | 734.00 | 30.56 | 567.45 | 66.93 |
| 56 | 1120.03 | 73.04 | 748.46 | 110.68 | 1042.80 | 43.42 | 826.84 | 97.53 |
| 59 | 1457.01 | 95.02 | 687.78 | 101.71 | 957.11 | 39.85 | 737.29 | 86.97 |
| 63 | 1703.79 | 111.12 | 788.70 | 116.63 | 1141.47 | 47.53 | 856.66 | 101.05 |
| 66 | 1651.37 | 107.70 | 1032.63 | 152.70 | 1584.30 | 65.97 | 1220.36 | 143.95 |
| 70 | 1796.39 | 123.14 | 1346.37 | 199.10 | 1813.35 | 75.51 | 1399.11 | 166.60 |
| 73 | 2361.95 | 164.27 | 1605.96 | 237.49 | 2251.14 | 93.74 | 1630.89 | 195.41 |
| 77 | | | 1795.31 | 265.49 | | | 1729.11 | 211.36 |

Amino Acid and Nucleotide Sequences

SEQ ID NO: 1 (human Tim-3) (Homo Sapiens)
MFSHLPFDCVLLLLLLLLTRSSEVEYRAEVGQNAYLPCFYTPAAPGNLVPVCWG
KGACPVFECGNVVLRTDERDVNYWTSRYWLNGDFRKGDVSLTIENVTLADSGIY
CCRIQIPGIMNDEKFNLKLVIK
SEQ ID NO: 2 (HCDR1 of Antibody A) (Artificial Sequence)
AASGFTFSSYYMS
SEQ ID NO: 3 (HCDR2 of Antibody A) (Artificial Sequence)
AISGSGGSTYYADSVKG
SEQ ID NO: 4 (HCDR3 of Antibody A) (Artificial Sequence)
ARYARTAFDL
SEQ ID NO: 5 (LCDR1 of Antibody A) (Artificial Sequence)
QASQDIYNYLN
SEQ ID NO: 6 (LCDR2 of Antibody A) (Artificial Sequence)
YAASSLQS
SEQ ID NO: 7 (LCDR3 of Antibody A) (Artificial Sequence)
QQANSFPPT
SEQ ID NO: 8 (HCVR of Antibody A) (Artificial Sequence)
EVQLLESGGGLVQPGGSLRLSCAASGFTFSSYYMSWVRQAPGKGLEWVSAISGS
GGSTYYADSVKGRFTISRDNSKNTLYLQMNSLRAEDTAVYYCARYARTAFDLW
GQGTLVTVSS
SEQ ID NO: 9 (LCVR of Antibody A) (Artificial Sequence)
DIVMTQSPSSLSASVGDGVTITCQASQDIYNYLNWYQQKPGKAPKLLIYAASSLQ
SGVPSRFSGSGSGTDFTLTISSLQPEDFATYYCQQANSFPPTFGQGTKLEIK
SEQ ID NO: 10 (HC of Antibody A) (Artificial Sequence)
EVQLLESGGGLVQPGGSLRLSCAASGFTFSSYYMSWVRQAPGKGLEWVSAISGS
GGSTYYADSVKGRFTISRDNSKNTLYLQMNSLRAEDTAVYYCARYARTAFDLW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPK
SCDKTHTCPPCPAPEAEGAPSVFLFPPKPKDTLMISRTPEVTCVVVDVSHEDPEVK
FNWYVDGVEVHNAKTKPREEQYNSTYRVVSVLTVLHQDWLNGKEYKCKVSNK
ALPSSIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHY
TQKSLSLSPGK
SEQ ID NO: 11 (LC of Antibody A) (Artificial Sequence)
DIVMTQSPSSLSASVGDGVTITCQASQDIYNYLNWYQQKPGKAPKLLIYAASSLQ
SGVPSRFSGSGSGTDFTLTISSLQPEDFATYYCQQANSFPPTFGQGTKLEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 12 (DNA of HC of Antibody A) (Artificial Sequence)
GAGGTGCAGCTGTTGGAGTCTGGCGGAGGGCTGGTGCAGCCGGGAGGCAGCC
TCAGGCTGAGCTGCGCTGCGAGCGGGTTTACTTTCTCGTCGTACTATATGTCG
TGGGTGAGACAAGCACCAGGTAAAGGACTTGAGTGGGTGTCCGCTATCTCAG
GCAGCGGAGGATCCACCTACTACGCGGATTCAGTCAAGGGAAGATTCACTAT
CTCGCGCGACAATTCCAAGAACACCCTGTACCTCCAGATGAACTCGCTGCGG
GCAGAAGATACGGCCGTGTACTACTGTGCCCGCTACGCCCGGACCGCCTTCG
ACTTGTGGGGTCAGGGAACCCTGGTCACTGTCTCCTCAGCTAGCACCAAGGG
CCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTC
GTGGAACTCAGGCGCACTGACCAGCGGCGTGCACACCTTCCCGGCTGTCCTA
CAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACC
AAGGTGGACAAGAGAGTTGAGCCCAAATCTTGTGACAAAACTCACACATGCC
CACCGTGCCCAGCACCTGAAGCCGAGGGGGCACCGTCAGTCTTCCTCTTCCCC
CCAAAACCCAAGGACACCCTCATGATCTCCCGGACCCCTGAGGTCACATGCG
TGGTGGTGGACGTGAGCCACGAAGACCCTGAGGTCAAGTTCAACTGGTATGT
GGACGGCGTGGAGGTGCATAATGCCAAGACAAAGCCGCGGGAGGAGCAGTA
CAACAGCACGTACCGTGTGGTCAGCGTCCTCACCGTCCTGCACCAAGACTGG
CTGAATGGCAAGGAGTACAAGTGCAAGGTCTCCAACAAAGCCCTCCCATCCT
CCATCGAGAAAACCATCTCCAAAGCCAAAGGGCAGCCCCGAGAACCACAGGT
GTACACCCTGCCCCCATCCCGGGAGGAGATGACCAAGAACCAAGTCAGCCTG
ACCTGCCTGGTCAAAGGCTTCTATCCCAGCGACATCGCCGTGGAGTGGGAGA
GCAATGGGCAGCCGGAGAACAACTACAAGACCACGCCTCCCGTGCTGGACTC
CGACGGCTCCTTCTTCCTCTATTCCAAGCTCACCGTGGACAAGAGCAGGTGGC
AGCAGGGGAACGTCTTCTCATGCTCCGTGATGCATGAGGCTCTGCACAACCA
CTACACGCAGAAGAGCCTCTCCCTGTCTCCGGGCAAA
SEQ ID NO: 13 (DNA of LC of Antibody A) (Artificial Sequence)
GACATCGTGATGACTCAAAGCCCTTCAAGCCTCTCGGCGTCAGTCGGTGATGG
CGTGACCATTACCTGTCAAGCATCCCAAGACATCTACAACTACTTGAATTGGT
ACCAGCAGAAGCCAGGGAAAGCCCCGAAGCTGCTGATCTACGCCGCCTCCTC
ACTTCAGAGCGGAGTGCCATCCCGCTTTTCCGGATCGGGGAGCGGAACGGAT
TTCACTCTGACCATCTCGTCGCTGCAACCGGAGGACTTCGCGACTTACTATTG
CCAGCAGGCTAACTCGTTCCCGCCCACTTTCGGACAGGGCACCAAGCTCGAA
ATCAAACGAACTGTGGCTGCACCATCTGTCTTCATCTTCCCGCCATCTGATGA
GCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCA
ACAGGGGAGAGTGT
SEQ ID NO: 14 (Human CEACAM1) (Homo Sapiens)
MGHLSAPLHRVRVPWQGLLLTASLLTFWNPPTTAQLTTESMPFNVAEGKEVLLL
VHNLPQQLFGYSWYKGERVDGNRQIVGYAIGTQQATPGPANSGRETIYPNASLLI
QNVTQNDTGFYTLQVIKSDLVNEEATGQFHVYPELPKPSISSNNSNPVEDKDAVA
FTCEPETQDTTYLWWINNQSLPVSPRLQLSNGNRTLTLLSVTRNDTGPYECEIQNP
VSANRSDPVTLNVTYGPDTPTISPSDTYYRPGANLSLSCYAASNPPAQYSWLINGT
FQQSTQELFIPNITVNNSGSYTCHANNSVTGCNRTTVKTIIVTELSPVVAKPQIKAS
KTTVTGDKDSVNLTCSTNDTGISIRWFFKNQSLPSSERMKLSQGNTTLSINPVKRE
DAGTYWCEVFNPISKNQSDPIMLNVNYNALPQENGLSPGAIAGIVIGVVALVALI
AVALACFLHFGKTGRASDQRDLTEHKPSVSNHTQDHSNDPPNKMNEVTYSTLNF
EAQQPTQPTSASPSLTATEIIYSEVKKQ
SEQ ID NO: 15 (Human Galectin-9) (Homo Sapiens)
MAFSGSQAPYLSPAVPFSGTIQGGLQDGLQITVNGTVLSSSGTRFAVNFQTGFSGN
DIAFHFNPRFEDGGYVVCNTRQNGSWGPEERKTHMPFQKGMPFDLCFLVQSSDF
KVMVNGILFVQYFHRVPFHRVDTISVNGSVQLSYISFQPPGVWPANPAPITQTVIH
TVQSAPGQMFSTPAIPPMMYPHPAYPMPFITTILGGLYPSKSILLSGTVLPSAQRFH
INLCSGNHIAFHLNPRFDENAVVRNTQIDNSWGSEERSLPRKMPFVRGQSFSVWIL
CEAHCLKVAVDGQHLFEYYHRLRNLPTINRLEVGGDIQLTHVQT
SEQ ID NO: 16 (Human PD-L1) (Homo Sapiens)
MRIFAVFIFMTYWHLLNAFTVTVPKDLYVVEYGSNMTIECKFPVEKQLDLAALIV
YWEMEDKNIIQFVHGEEDLKVQHSSYRQRARLLKDQLSLGNAALQITDVKLQDA
GVYRCMISYGGADYKRITVKVNAPYNKINQRILVVDPVTSEHELTCQAEGYPKA
EVIWTSSDHQVLSGKTTTTNSKREEKLFNVTSTLRINTTTNEIFYCTFRRLDPEENH
TAELVIPELPLAHPPNERTHLVILGAILLCLGVALTFIFRLRKGRMMDVKKCGIQD
TNSKKQSDTHLEET
SEQ ID NO: 17 (HCDR1 of Antibody B) (Artificial Sequence)
KASGGTFSSYAIS
SEQ ID NO: 18 (HCDR2 of Antibody B) (Artificial Sequence)
GIIPIFGTANYAQKFQG
SEQ ID NO: 19 (HCDR3 of Antibody B) (Artificial Sequence)
ARSPDYSPYYYYGMDV
SEQ ID NO: 20 (LCDR1 of Antibody B) (Artificial Sequence)
SGSSSNIGSNTVN
SEQ ID NO: 21 (LCDR2 of Antibody B) (Artificial Sequence)
YGNSNRPS
SEQ ID NO: 22 (LCDR3 of Antibody B) (Artificial Sequence)
QSYDSSLSGSV
SEQ ID NO: 23 (HCVR of Antibody B) (Artificial Sequence)
QVQLVQSGAEVKKPGSSVKVSCKASGGTFSSYAISWVRQAPGQGLEWMGGIIPIF
GTANYAQKFQGRVTITADKSTSTAYMELSSLRSEDTAVYYCARSPDYSPYYYYG
MDVWGQGTTVTVSS
SEQ ID NO: 24 (LCVR of Antibody B) (Artificial Sequence)
QSVLTQPPSASGTPGQRVTISCSGSSSNIGSNTVNWYQQLPGTAPKLLIYGNSNRP
SGVPDRFSGSKSGTSASLAISGLQSEDEADYYCQSYDSSLSGSVFGGGIKLTVLG
SEQ ID NO: 25 (HC of Antibody B) (Artificial Sequence)
QVQLVQSGAEVKKPGSSVKVSCKASGGTFSSYAISWVRQAPGQGLEWMGGIIPIF
GTANYAQKFQGRVTITADKSTSTAYMELSSLRSEDTAVYYCARSPDYSPYYYYG
MDVWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KRVEPKSCDKTHTCPPCPAPEAEGAPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPSSIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHE
ALHNHYTQKSLSLSPGK
SEQ ID NO: 26 (LC of Antibody B) (Artificial Sequence)
QSVLTQPPSASGTPGQRVTISCSGSSSNIGSNTVNWYQQLPGTAPKLLIYGNSNRP
SGVPDRFSGSKSGTSASLAISGLQSEDEADYYCQSYDSSLSGSVFGGGIKLTVLGQ
PKAAPSVTLFPPSSEELQANKATLVCLISDFYPGAVTVAWKADSSPVKAGVETTT
PSKQSNNKYAASSYLSLTPEQWKSHRSYSCQVTHEGSTVEKTVAPAECS
SEQ ID NO: 27 (DNA of HC of Antibody B) (Artificial Sequence)
CAGGTCCAGCTGGTCCAGTCAGGGGCCGAGGTCAAAAAGCCAGGGTCATCTG
TCAAAGTGTCTTGTAAGGCATCCGGGGGCACATTTTCCAGCTACGCTATCTCC
TGGGTGAGACAGGCACCAGGGCAGGGTCTGGAGTGGATGGGCGGAATCATTC
CCATCTTCGGGACCGCCAACTACGCTCAGAAGTTTCAGGGAAGGGTCACTATT
ACCGCCGACAAAAGCACATCTACTGCTTATATGGAGCTGTCTAGTCTGAGGTC
TGAAGATACCGCAGTGTACTATTGCGCCCGGAGTCCCGACTATAGCCCTTACT
ATTACTATGGCATGGATGTCTGGGGCCAGGGAACCACAGTGACAGTCTCATC
CGCTAGCACCAAGGGCCCATCGGTCTTCCCCCTGGCACCCTCCTCCAAGAGCA
CCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACA
AAACTCACACATGCCCACCGTGCCCAGCACCTGAAGCCGAGGGGGCACCGTC
AGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAA
GTTCAACTGGTATGTGGACGGCGTGGAGGTGCATAATGCCAAGACAAAGCCG
CGGGAGGAGCAGTACAACAGCACGTACCGTGTGGTCAGCGTCCTCACCGTCC
TGCACCAAGACTGGCTGAATGGCAAGGAGTACAAGTGCAAGGTCTCCAACAA
AGCCCTCCCATCCTCCATCGAGAAAACCATCTCCAAAGCCAAAGGGCAGCCC
CGAGAACCACAGGTGTACACCCTGCCCCCATCCCGGGAGGAGATGACCAAGA
ACCAAGTCAGCCTGACCTGCCTGGTCAAAGGCTTCTATCCCAGCGACATCGCC
GTGGAGTGGGAGAGCAATGGGCAGCCGGAGAACAACTACAAGACCACGCCT
CCCGTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGCAAA
SEQ ID NO: 28 (DNA of LC of Antibody B) (Artificial Sequence)
CAGTCCGTCCTGACACAGCCACCCTCAGCCTCTGGCACCCCTGGGCAGCGAGT
GACAATCTCTTGTTCTGGGAGTTCCTCAAATATTGGTAGTAACACCGTGAATT
GGTACCAGCAGCTGCCCGGCACAGCACCTAAGCTGCTGATCTATGGAAACTC
AAATAGGCCATCCGGAGTCCCCGACCGGTTCTCTGGTAGTAAATCAGGCACTT
CCGCCAGCCTGGCTATTAGCGGGCTGCAGTCTGAGGACGAAGCCGATTACTA
TTGCCAGTCTTACGATTCCAGCCTGTCTGGAAGTGTGTTTGGCGGAGGGATCA
AGCTGACCGTCCTGGGCCAGCCTAAGGCTGCCCCCTCGGTCACTCTGTTCCCG
CCCTCCTCTGAGGAGCTTCAAGCCAACAAGGCCACACTGGTGTGTCTCATAAG
TGACTTCTACCCGGGAGCCGTGACAGTGGCCTGGAAGGCAGATAGCAGCCCC
GTCAAGGCGGGAGTGGAGACCACCACACCCTCCAAACAAAGCAACAACAAG
TACGCGGCCAGCAGCTACCTGAGCCTGACGCCTGAGCAGTGGAAGTCCCACA
GAAGCTACAGCTGCCAGGTCACGCATGAAGGGAGCACCGTGGAGAAGACAG
TGGCCCCTGCAGAATGCTCT
SEQ ID NO: 29 (Atezolizumab LC) (Artificial Sequence)
DIQMTQSPSSLSASVGDRVTITCRASQDVSTAVAWYQQKPGKAPKLLIYSASFLY
SGVPSRFSGSGSGTDFTLTISSLQPEDFATYYCQQYLYHPATFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 30 (Atezolizumab HC) (Artificial Sequence)
EVQLVESGGGLVQPGGSLRLSCAASGFTFSDSWIHWVRQAPGKGLEWVAWISPY
GGSTYYADSVKGRFTISADTSKNTAYLQMNSLRAEDTAVYYCARRHWPGGFDY
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPE
VKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 31 (Durvalumab LC) (Artificial Sequence)
EIVLTQSPATLSLSPGERATLSCRASQSVSSYLAWYQQKPGQAPRLLIYDASNRAT
GIPARFSGSGSGTDFTLTISSLEPEDFAVYYCQQSSNWPRTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 32 (Durvalumab HC) (Artificial Sequence)
QVQLVESGGGVVQPGRSLRLDCKASGITFSNSGMHWVRQAPGKGLEWVAVIWY
DGSKRYYADSVKGRFTISRDNSKNTLFLQMNSLRAEDTAVYYCATNDDYWGQG
TLVTVSSASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVESKYGP
PCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSL
SLGK
SEQ ID NO: 33 (Avelumab LC) (Artificial Sequence)
QSALTQPASVSGSPGQSITISCTGTSSDVGGYNYVSWYQQHPGKAPKLMIYDVSN
RPSGVSNRFSGSKSGNTASLTISGLQAEDEADYYCSSYTSSSTRVFGTGTKVTVLG
QPKANPTVTLFPPSSEELQANKATLVCLISDFYPGAVTVAWKADGSPVKAGVETT
KPSKQSNNKYAASSYLSLTPEQWKSHRSYSCQVTHEGSTVEKTVAPTECS
SEQ ID NO: 34 (Avelumab HC) (Artificial Sequence)
EVQLLESGGGLVQPGGSLRLSCAASGFTFSSYIIVIMWVRQAPGKGLEWVSSIYPSG
GITFYADTVKGRFTISRDNSKNTLYLQMNSLRAEDTAVYYCARIKLGTVTTVDY
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 35 (BMS-936559 LCVR) (Artificial Sequence)
EIVLTQSPATLSLSPGERATLSCRASQSVSSYLAWYQQKPGQAPRLLIYDASNRAT
GIPARFSGSGSGTDFTLTISSLEPEDFAVYYCQQRSNWPTFGQGTKVEIK
SEQ ID NO: 36 (BMS-936559 HCVR) (Artificial Sequence)
QVQLVQSGAEVKKPGSSVKVSCKTSGDTFSTYAISWVRQAPGQGLEWMGGIIPIF
GKAHYAQKFQGRVTITADESTSTAYMELSSLRSEDTAVYFCARKFHFVSGSPFG
MDVWGQGTTVTVSS

We claim:

1. A method of treating cancer comprising administering to a patient in need, thereof an effective amount of an anti-human Tim-3 (SEQ ID NO: 1) antibody in simultaneous, separate, or sequential combination with an effective amount of an anti-human PD-L1 (SEQ ID NO: 16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7.

2. The method of claim 1, wherein the anti-human Tim-3 antibody comprises a heavy chain variable region having the amino acid sequence of SEQ ID NO: 8 and a light chain variable region having the amino acid sequence of SEQ ID NO: 9.

3. The method of claim 2, wherein the anti-human Tim-3 antibody comprises a heavy chain having the amino acid sequence of SEQ ID NO: 10 and a light chain having the amino acid sequence of SEQ ID NO: 11.

4. The method of any one of claims 1-3, wherein the anti-human PD-L1 antibody is atezolizumab, durvalumab, or avelumab.

5. The method of any one of claims 1-3, wherein the anti-human PD-L1 antibody comprises HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22.

6. The method of claim 5, wherein the anti-human PD-L1 antibody comprises a heavy chain variable region having the amino acid sequence of SEQ ID NO: 23 and a light chain variable region having the amino acid sequence of SEQ ID NO: 24.

7. The method of claim 6, wherein the anti-human PD-L1 antibody comprises a heavy chain having the amino acid sequence of SEQ ID NO: 25 and a light chain having the amino acid sequence of SEQ ID NO: 26.

8. The method of any one of claims 1-7, wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

9. The method of claim 8, wherein the lung cancer is non-small cell lung cancer.

10. The method of any one of claims 1-9, wherein at least one of the anti-human Tim-3 antibody and anti-human PD-L1 antibody is administered in simultaneous, separate, or sequential combination with ionizing radiation.

11. The method of any one of claims 1-10, wherein at least one of the anti-human Tim-3 antibody and anti-human PD-L1 antibody is administered in simultaneous, separate, or sequential combination with one or more chemotherapeutic agents.

12. An anti-human Tim-3 (SEQ ID NO: 1) antibody for use in simultaneous, separate, or sequential combination with an anti-human PD-L1 (SEQ ID NO: 16) antibody, in the treatment of cancer, wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO: 5, LCDR2 having the amino acid sequence of SEQ ID NO: 6, and LCDR3 having the amino acid sequence of SEQ ID NO: 7.

13. The anti-human Tim-3 antibody for use of claim 12, wherein the anti-human Tim-3 antibody comprises a heavy chain variable region having the amino acid sequence of SEQ ID NO: 8 and a light chain variable region having the amino acid sequence of SEQ ID NO: 9.

14. The anti-human Tim-3 antibody for use of claim 13, wherein the anti-human Tim-3 antibody comprises a heavy chain having the amino acid sequence of SEQ ID NO: 10 and a light chain having the amino acid sequence of SEQ ID NO: 11.

15. The anti-human Tim-3 antibody for use of any one of claims 12-14, wherein the anti-human PD-L1 antibody is atezolizumab, durvalumab, or avelumab.

16. The anti-human Tim-3 antibody for use of any one of claims 12-14, wherein the anti-human PD-L1 antibody comprises HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22.

17. The anti-human Tim-3 antibody for use of claim 16, wherein the anti-human PD-L1 antibody comprises a heavy chain variable region having the amino acid sequence of SEQ ID NO: 23 and a light chain variable region having the amino acid sequence of SEQ ID NO: 24.

18. The anti-human Tim-3 antibody for use of claim 17, wherein the anti-human PD-L1 antibody comprises a heavy chain having the amino acid sequence of SEQ ID NO: 25 and a light chain having the amino acid sequence of SEQ ID NO: 26.

19. The anti-human Tim-3 antibody for use of any one of claims 12-18, wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

20. The anti-human Tim-3 antibody for use of claim 19, wherein the lung cancer is non-small cell lung cancer.

21. The anti-human Tim-3 antibody for use of any one of claims 12-20, wherein at least one of the anti-human Tim-3 antibody and anti-human PD-L1 antibody is administered in simultaneous, separate, or sequential combination with ionizing radiation.

22. The anti-human Tim-3 antibody for use of any one of claims 12-21, wherein at least one of the anti-human Tim-3 antibody and anti-human PD-L1 antibody is administered in simultaneous, separate, or sequential combination with one or more chemotherapeutic agents.

23. Use of an anti-human Tim-3 (SEQ ID NO:1) antibody for the manufacture of a medicament for the treatment of cancer, wherein the medicament is to be administered simultaneously, separately, or sequentially with an anti-human PD-L1 (SEQ ID NO: 16) antibody; wherein the anti-human Tim-3 antibody comprises HCDR1 having the amino acid sequence of SEQ ID: 2, HCDR2 having the amino acid sequence of SEQ ID NO: 3, HCDR3 having the amino acid sequence of SEQ ID NO: 4, LCDR1 having the amino acid sequence of SEQ ID NO:5, LCDR2 having the amino acid sequence of SEQ ID NO:6, and LCDR3 having the amino acid sequence of SEQ ID NO:7.

24. The use of claim 23, wherein the anti-human Tim-3 antibody comprises a heavy chain variable region having the amino acid sequence of SEQ ID NO: 8 and a light chain variable region having the amino acid sequence of SEQ ID NO: 9.

25. The use of claim 24, wherein the anti-human Tim-3 antibody comprises a heavy chain having the amino acid sequence of SEQ ID NO: 10 and a light chain having the amino acid sequence of SEQ ID NO: 11.

26. The use of any one of claims 23-25, wherein the anti-human PD-L1 antibody is atezolizumab, durvalumab, or avelumab.

27. The use of any one of claims 23-25, wherein the anti-human PD-L1 antibody comprises HCDR1 having the amino acid sequence of SEQ ID: 17, HCDR2 having the amino acid sequence of SEQ ID NO: 18, HCDR3 having the amino acid sequence of SEQ ID NO: 19, LCDR1 having the amino acid sequence of SEQ ID NO: 20, LCDR2 having the amino acid sequence of SEQ ID NO: 21, and LCDR3 having the amino acid sequence of SEQ ID NO: 22.

28. The use of claim 27, wherein the anti-human PD-L1 antibody comprises a heavy chain variable region having the amino acid sequence of SEQ ID NO: 23 and a light chain variable region having the amino acid sequence of SEQ ID NO: 24.

29. The use of claim 28, wherein the anti-human PD-L1 antibody comprises a heavy chain having the amino acid sequence of SEQ ID NO: 25 and a light chain having the amino acid sequence of SEQ ID NO: 26.

30. The use of any one of claims 23-29, wherein the cancer is melanoma, lung cancer, head and neck cancer, colorectal cancer, pancreatic cancer, gastric cancer, kidney cancer, bladder cancer, prostate cancer, breast cancer, ovarian cancer, esophageal cancer, soft tissue sarcoma, or liver cancer.

31. The use of claim 30, wherein the lung cancer is non-small cell lung cancer.

32. The use of any one of claims 23-31, wherein at least one of the anti-human Tim-3 antibody and anti-human PD-L1 antibody is administered in simultaneous, separate, or sequential combination with ionizing radiation.

33. The use of any one of claims 23-32, wherein at least one of the anti-human Tim-3 antibody and anti-human PD-L1 antibody is administered in simultaneous, separate, or sequential combination with one or more chemotherapeutic agents.


SEQUENCE LISTING (36 sequences)

SEQ ID NO: 1 - protein, 130 aa, Human
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 2 - protein, 13 aa, Artificial Sequence
  Synthetic Construct
AASGFTFSSYYMS

SEQ ID NO: 3 - protein, 17 aa, Artificial Sequence
  Synthetic Construct
AISGSGGSTYYADSVKG

SEQ ID NO: 4 - protein, 10 aa, Artificial Sequence
  Synthetic Construct
ARYARTAFDL

SEQ ID NO: 5 - protein, 11 aa, Artificial Sequence
  Synthetic Construct
QASQDIYNYLN

SEQ ID NO: 6 - protein, 8 aa, Artificial Sequence
  Synthetic Construct
YAASSLQS

SEQ ID NO: 7 - protein, 9 aa, Artificial Sequence
  Synthetic Construct
QQANSFPPT

SEQ ID NO: 8 - protein, 117 aa, Artificial Sequence
  Synthetic Construct
EVQLLESGGGLVQPGGSLRLSCAASGFTFSSYYMSWVRQAPGKGLEWVSAISGSGGSTYYADSVKGRFTISRDNSKNTLYLQMNSLRAEDTAVYYCARYARTAFDLWGQGTLVTVSS

SEQ ID NO: 9 - protein, 107 aa, Artificial Sequence
  Synthetic Construct
DIVMTQSPSSLSASVGDGVTITCQASQDIYNYLNWYQQKPGKAPKLLIYAASSLQSGVPSRFSGSGSGTDFTLTISSLQPEDFATYYCQQANSFPPTFGQGTKLEIK

SEQ ID NO: 10 - protein, 447 aa, Artificial Sequence
  Synthetic Construct
EVQLLESGGGLVQPGGSLRLSCAASGFTFSSYYMSWVRQAPGKGLEWVSAISGSGGSTYYADSVKGRFTISRDNSKNTLYLQMNSLRAEDTAVYYCARYARTAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  Synthetic Construct
DIVMTQSPSSLSASVGDGVTITCQASQDIYNYLNWYQQKPGKAPKLLIYAASSLQSGVPSRFSGSGSGTDFTLTISSLQPEDFATYYCQQANSFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 1341 bp, Artificial Sequence
  Synthetic Construct
gaggtgcagctgttggagtctggcggagggctggtgcagccgggaggcagcctcaggctgagctgcgctgcgagcgggtttactttctcgtcgtactatatgtcgtgggtgagacaagcaccaggtaaaggacttgagtgggtgtccgctatctcaggcagcggaggatccacctactacgcggattcagtcaagggaagattcactatctcgcgcgacaattccaagaacaccctgtacctccagatgaactcgctgcgggcagaagatacggccgtgtactactgtgcccgctacgcccggaccgccttcgacttgtggggtcagggaaccctggtcactgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 13 - DNA, 642 bp, Artificial Sequence
  Synthetic Construct
gacatcgtgatgactcaaagcccttcaagcctctcggcgtcagtcggtgatggcgtgaccattacctgtcaagcatcccaagacatctacaactacttgaattggtaccagcagaagccagggaaagccccgaagctgctgatctacgccgcctcctcacttcagagcggagtgccatcccgcttttccggatcggggagcggaacggatttcactctgaccatctcgtcgctgcaaccggaggacttcgcgacttactattgccagcaggctaactcgttcccgcccactttcggacagggcaccaagctcgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 526 aa, Human
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 15 - protein, 323 aa, Human
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 16 - protein, 290 aa, Human
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - protein, 13 aa, Artificial Sequence
  Synthetic Construct
KASGGTFSSYAIS

SEQ ID NO: 18 - protein, 17 aa, Artificial Sequence
  Synthetic Construct
GIIPIFGTANYAQKFQG

SEQ ID NO: 19 - protein, 16 aa, Artificial Sequence
  Synthetic Construct
ARSPDYSPYYYYGMDV

SEQ ID NO: 20 - protein, 13 aa, Artificial Sequence
  Synthetic Construct
SGSSSNIGSNTVN

SEQ ID NO: 21 - protein, 8 aa, Artificial Sequence
  Synthetic Construct
YGNSNRPS

SEQ ID NO: 22 - protein, 11 aa, Artificial Sequence
  Synthetic Construct
QSYDSSLSGSV

SEQ ID NO: 23 - protein, 123 aa, Artificial Sequence
  Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSS

SEQ ID NO: 24 - protein, 111 aa, Artificial Sequence
  Synthetic Construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLG

SEQ ID NO: 25 - protein, 453 aa, Artificial Sequence
  Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 216 aa, Artificial Sequence
  Synthetic Construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 27 - DNA, 1359 bp, Artificial Sequence
  Synthetic Construct
caggtccagctggtccagtcaggggccgaggtcaaaaagccagggtcatctgtcaaagtgtcttgtaaggcatccgggggcacattttccagctacgctatctcctgggtgagacaggcaccagggcagggtctggagtggatgggcggaatcattcccatcttcgggaccgccaactacgctcagaagtttcagggaagggtcactattaccgccgacaaaagcacatctactgcttatatggagctgtctagtctgaggtctgaagataccgcagtgtactattgcgcccggagtcccgactatagcccttactattactatggcatggatgtctggggccagggaaccacagtgacagtctcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 28 - DNA, 648 bp, Artificial Sequence
  Synthetic Construct
cagtccgtcctgacacagccaccctcagcctctggcacccctgggcagcgagtgacaatctcttgttctgggagttcctcaaatattggtagtaacaccgtgaattggtaccagcagctgcccggcacagcacctaagctgctgatctatggaaactcaaataggccatccggagtccccgaccggttctctggtagtaaatcaggcacttccgccagcctggctattagcgggctgcagtctgaggacgaagccgattactattgccagtcttacgattccagcctgtctggaagtgtgtttggcggagggatcaagctgaccgtcctgggccagcctaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgctct

SEQ ID NO: 29 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 448 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 440 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 34 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 106 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 36 - protein, 123 aa, Artificial Sequence
  Synthetic Construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS